(19) United States
(12) Patent Application Publication
(10) Pub. No.: US 2023/0150170 A1
(43) Pub. Date: May 18, 2023

(54) PROBE AND METHOD FOR MONITORING FRESH CONCRETE USING AN ELECTROMECHANICAL ACTUATOR

(71) Applicant: COMMAND ALKON INCORPORATED, Birmingham,, AL (US)

(72) Inventors: Denis BEAUPRE, Québec (CA); Jerome CHAPDELAINE, Québec (CA); Robin JEAN, Québec (CA)

(73) Assignee: COMMAND ALKON INCORPORATED, Birmingham,, AL (US)

(21) Appl. No.: 17/908,160

(22) PCT Filed: Mar. 1, 2021
(86) PCT No.: PCT/US2021/020212

§ 371 Date: Aug. 30, 2022

Related U.S. Application Data
(60) Provisional application No. 62/983,949, filed on Mar. 2, 2020.

(51) Int. Cl. B28C 7/02; B28C 5/42; G01N 9/16; G01N 33/38

(52) U.S. Cl. CPC B28C 7/024; B28C 5/422; G01N 9/16; G01N 33/383; G01N 2011/0046

(57) ABSTRACT

There is described a probe for monitoring fresh concrete received in a drum of a fresh concrete mixer. The probe generally has an electromechanical actuator having a frame mounted within the drum and a moving element actuatably mounted to the frame, the moving element having a fresh concrete interface exposed within the drum and experiencing a resistance to movement within the drum upon actuation of the electromechanical actuator with an electrical signal; and a measurement unit measuring a resistance response during the actuation and generating a response signal based on the measured resistance response, the generated response signal comprising monitoring Information concerning the fresh concrete within the drum, if any.

### FIELD

The improvements generally relate to handling fresh concrete received in a drum of a fresh concrete mixer, and more specifically relate to measuring information concerning the fresh concrete as the drum rotates.

### BACKGROUND

Fresh concrete is formed of a mixture of ingredients including at least cement-based material and water in given proportions. The ingredients are typically transported inside a drum of mixer truck where the fresh concrete can be mixed prior to pouring thereof.

Even though mixer trucks can extend the life of fresh concrete, they do not make the fresh concrete last indefinitely. Typically, properties of the fresh concrete in the concrete mixer such as viscosity, yield, slump, air content and density can vary over time. The volume of fresh concrete received within the drum can also change, as it is also usual for mixer trucks to perform partial discharges on the go. As informed decision on how to handle the fresh concrete should be made based on measured information, there exists probes specifically designed for mixer trucks. Examples of such probes are described in U.S. Pat. No. 10,156,547 B2 and in published international patent application no. PCT/IB2010/054542, to name a few examples.

Although existing probes for mixer trucks or other fresh concrete mixers are satisfactory to a certain degree, there remains room for improvement.

### SUMMARY

In an aspect, there is described a probe for monitoring fresh concrete received in a drum of a fresh concrete mixer such as a mixer truck, for instance. The probe generally has an electromechanical actuator with a frame mounted within the drum and a moving element actuatably mounted to the frame. The moving element has a fresh concrete interface which is exposed within the drum and which experiences a resistance to movement within the drum upon actuation of the electromechanical actuator with an electrical signal. The resistance experienced by the fresh concrete interface during the actuation can be stronger in presence of fresh concrete, weaker in presence of water and weakest in presence of air. A measurement unit is also provided. During use, the measurement circuit measures a resistance response during the actuation of the moving element and generates a response signal based on the measured resistance response. It is intended that the generated response signal has monitoring information concerning the fresh concrete and/or water within the drum, if any. In some embodiments, the measurement circuit includes an accelerometer measuring a mechanical response of the fresh concrete interface in which case the measured resistance response is mechanical. Additionally or alternately, the measurement circuit includes a power meter measuring the amount of electrical power consumed by the electromechanical during the actuation in which case the measured resistance response is electrical.

In accordance with a first aspect of the present disclosure, there is provided a probe for monitoring fresh concrete received in a drum of a fresh concrete mixer, the probe comprising: an electromechanical actuator having a frame mounted within the drum and a moving element actuatably mounted to the frame, the moving element having a fresh concrete interface exposed within said drum and experiencing a resistance to movement within said drum upon actuation of the electromechanical actuator with an electrical signal; and a measurement unit measuring a resistance response during said actuation and generating a response signal based on said measured resistance response, the generated response signal comprising monitoring information concerning the fresh concrete within the drum, if any.

Further in accordance with the first aspect of the present disclosure, the frame can for example be a housing enclosing the moving element, the housing can for example have at least a given wall with an inner side mechanically coupled to the moving element and an outer side acting as the fresh concrete interface.

Still further in accordance with the first aspect of the present disclosure, the given wall can for example be provided in the form of a membrane having a thickness below a given thickness threshold.

Still further in accordance with the first aspect of the present disclosure, the measurement unit can for example have an electrical response sensor measuring an electrical response of said electromechanical actuator during said actuation.

Still further in accordance with the first aspect of the present disclosure, the electrical response sensor can for example have an electrical power meter measuring an electrical power value indicative of an electrical power consumed by said electromechanical actuator during said actuation.

Still further in accordance with the first aspect of the present disclosure, the measurement unit can for example have a mechanical response sensor measuring a mechanical response of said electromechanical actuator during said actuation.

Still further in accordance with the first aspect of the present disclosure, the mechanical response sensor can for example have a position sensor measuring an amplitude value indicative of an amplitude of movement of said moving element during said actuation.

Still further in accordance with the first aspect of the present disclosure, the probe can for example further have a controller communicatively coupled to the measurement unit, the controller having a processor and a non-transitory memory having stored thereon instructions that when executed by the processor performs the step of monitoring the fresh concrete received in the drum based on said generated response signal.

Still further in accordance with the first aspect of the present disclosure, said actuation and measurement can for example be performed a plurality of times during at least a rotation of the drum, said monitoring can for example include determining a volume of the fresh concrete inside the drum based on said resistance responses experienced during the at least the rotation of the drum.

Still further in accordance with the first aspect of the present disclosure, said monitoring can for example include determining a rheological property of said fresh concrete, said rheological property can for example be selected in a group of rheological properties including viscosity, yield and slump.

Still further in accordance with the first aspect of the present disclosure, said monitoring can for example include determining a physical property of said fresh concrete, said physical property can for example be selected in a group of physical properties including air content and density.

Still further in accordance with the first aspect of the present disclosure, said monitoring can for example be based on calibration data pertaining to different resistance responses as function of different properties of the fresh concrete.

Still further in accordance with the first aspect of the present disclosure, said electrical signal is an oscillatory electrical signal having an amplitude oscillating over time, the resistance response experienced by the fresh concrete interface oscillating over time during said actuation with said oscillatory electrical signal.

Still further in accordance with the first aspect of the present disclosure, said oscillatory electrical signal can for example have a frequency ranging between about 20 Hz and about 20 kHz.

Still further in accordance with the first aspect of the present disclosure, the fresh concrete mixer can for example be a mixer truck.

In accordance with a second aspect of the present disclosure, there is provided a method of monitoring fresh concrete received in a drum of a fresh concrete mixer, the method comprising: exposing a fresh concrete interface within said drum; mechanically coupling a moving element of an electromechanical actuator to said fresh concrete interface; actuating the electromechanical actuator with an electrical signal, said actuating including moving said moving element relative to the fresh concrete interface, said moving element thereby experiencing a resistance to movement via said fresh concrete interface; measuring a resistance response during said actuating and generating a response signal based on said measured resistance response, the generated response signal comprising monitoring information concerning the fresh concrete within the drum, if any.

Further in accordance with the second aspect of the present disclosure, said measuring the resistance response can for example include measuring an electrical response of said electromechanical actuator during said actuation.

Still further in accordance with the second aspect of the present disclosure, said measuring the electrical response can for example include measuring an electrical power value indicative of an electrical power consumed by said electromechanical actuator during said actuation.

Still further in accordance with the second aspect of the present disclosure, said measuring the resistance response can for example include measuring a mechanical response of said electromechanical actuator during said actuation.

Still further in accordance with the second aspect of the present disclosure, said measuring the mechanical response can for example include measuring an amplitude value indicative of an amplitude of movement of said moving element during said actuation.

Still further in accordance with the second aspect of the present disclosure, the method can for example further comprise monitoring said fresh concrete based on the generated response signal.

Still further in accordance with the second aspect of the present disclosure, said actuating and said measuring can for example be performed a plurality of times during at least a rotation of the drum, said monitoring can for example include determining a volume of the fresh concrete inside the drum based on said resistance responses experienced during the at least the rotation of the drum.

Still further in accordance with the second aspect of the present disclosure, said monitoring can for example include determining a rheological property of said fresh concrete, said rheological property can for example be selected in a group of rheological properties including viscosity, yield and slump.

Still further in accordance with the second aspect of the present disclosure, said monitoring includes determining a physical property of said fresh concrete, said physical property can for example be selected in a group of physical properties including air content and density.

Still further in accordance with the second aspect of the present disclosure, said electrical signal can for example be an oscillatory electrical signal having an amplitude oscillating over time, said actuating can for example include moving said moving element against the fresh concrete interface in at least a back and forth sequence.

Still further in accordance with the second aspect of the present disclosure, the fresh concrete mixer can for example be a mixer truck.

Many further features and combinations thereof concerning the present improvements will appear to those skilled in the art following a reading of the instant disclosure.

### DESCRIPTION OF THE FIGURES

In the figures,

FIG. 1 is a schematic view of an example of a system for monitoring fresh concrete received in a drum of a mixer truck, with a probe mounted inside the drum and a controller, in accordance with one or more embodiments;

FIG. 2 is a sectional view of the drum of FIG. 1, taken along section 2-2 of FIG. 1, in accordance with one or more embodiments;

FIG. 3 is a block diagram of the system of FIG. 1, with the probe having an electromechanical actuator and a measurement unit measuring a mechanical response of the electromechanical actuator, in accordance with one or more embodiments;

FIG. 4 is a schematic view of an example of a computing device of the controller of FIG. 1, in accordance with one or more embodiments;

FIG. 5A is a sectional view of an example of a probe for monitoring fresh concrete received in a drum of a mixer truck, showing a housing having a given wall acting as a fresh concrete interface, in accordance with one or more embodiments;

FIG. 5B is a top view of the probe of FIG. 5A, in accordance with one or more embodiments;

FIG. 6 is a schematic view of a system incorporating the probe of FIG. 5A and a controller, in accordance with one or more embodiments;

FIG. 7A is a graph showing average accelerometer magnitude as function of a frequency of the oscillatory signal, in accordance with one or more embodiments;

FIG. 7B is a graph showing integral values of the average accelerometer magnitudes of FIG. 7A as integrated over a given frequency band, in accordance with one or more embodiments;

FIG. 8A is a graph showing average accelerometer magnitude as function of a frequency of the oscillatory signal, in accordance with one or more embodiments;

FIG. 8B is a graph showing integral values of the average accelerometer magnitudes of FIG. 8A as integrated over a given frequency band, in accordance with one or more embodiments; and

FIG. 9 is a block diagram of an example of a system for monitoring fresh concrete received in a drum of a mixer truck, with a probe having an electromechanical actuator and a measurement unit measuring an electrical response of the electromechanical actuator, in accordance with one or more embodiments.

### DETAILED DESCRIPTION

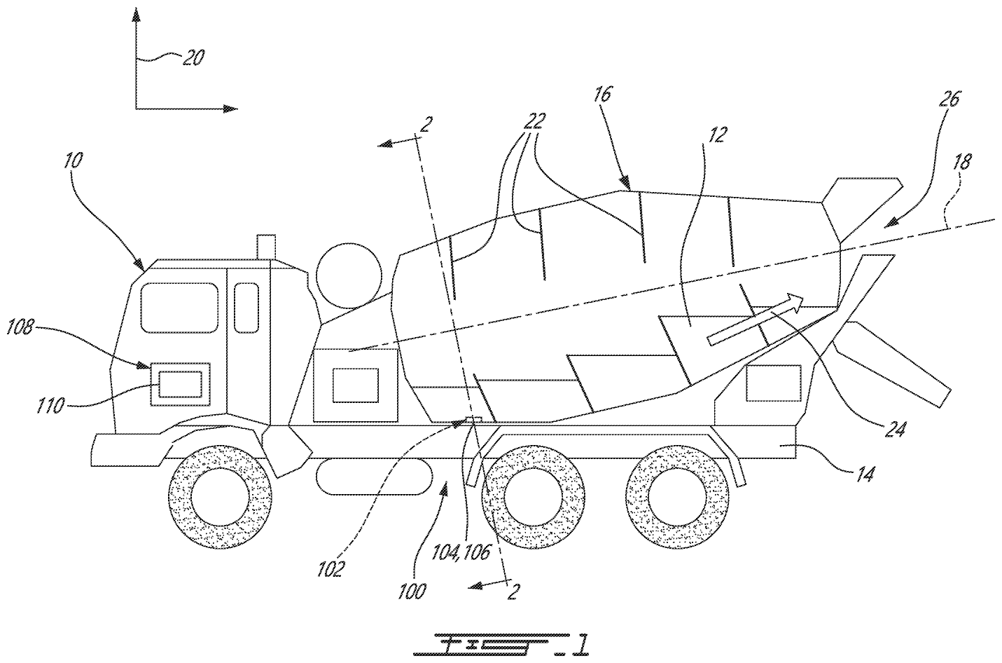

FIG. 1 shows an example of a fresh concrete mixer truck 10 (hereinafter referred to as “the mixer truck 10”) for handling fresh concrete 12. As shown, the mixer truck 10 has a truck frame 14 and a rotating drum 16 which is rotatably mounted to the truck frame 14. As such, the drum 16 can be rotated about a rotation axis 18 which is at least partially horizontally-oriented relative to the vertical 20.

As illustrated, the drum 16 has inwardly protruding blades 22 mounted inside the drum 16 which, when the drum 16 is rotated in an unloading direction, force the fresh concrete 12 along a discharge direction 24 towards a discharge outlet 26 of the drum 16 so as to be discharged at a job site, for instance. In contrast, when the drum 16 is rotated in a mixing direction, opposite to the unloading direction, the fresh concrete 12 is kept and mixed inside the drum 16.

In some embodiments, concrete constituents (e.g., cement, aggregate and water) are loaded in the drum 16 after which the drum 16 can be rotated a certain number of rotations in the mixing direction at a certain rotation speed so as to suitably mix the concrete constituents to one another, thus yielding the fresh concrete 12. In other embodiments, already mixed fresh concrete is loaded inside the drum 16, in which case the fresh concrete 12 can still be further mixed inside the drum 16 before discharge.

As shown, the mixer truck has a system 100 for monitoring the fresh concrete 12 received in the drum 16 of the mixer truck 10. As will be described below, the system 100 can be used to measure information pertaining to the fresh concrete 12 received in the drum 16. The measured information can then be used to handle the fresh concrete 12 satisfactorily. Examples of the information measured by the system 100 can include, but not limited to, physical properties (e.g., air content, density, temperature), rheological properties (e.g., viscosity, yield, slump), or other information concerning the fresh concrete 12 such as the volume of fresh concrete 12 received in the drum 16 at a given moment in time. Based on the monitored information, the fresh concrete 12 can be handled by, for instance, adding water into the drum 16, adding concrete constituents into the drum 16, adding adjuvant(s) in the drum 16, mixing the concrete constituents at a high speed range for a given of drum rotations, agitating the fresh concrete at a low speed range for a given period of time, and wholly or partially discharging the fresh concrete 12 at a job site.

As depicted in this embodiment, the system 100 has a probe 102 having an electromechanical actuator 104 actuatable within the fresh concrete 12 and a measurement unit 106 measuring a response of the electromechanical actuator 104 during actuation. The system 100 also incorporates a controller 108 communicatively coupled to the probe 102 for monitoring the fresh concrete 12 based on the measured response.

As illustrated, the controller 108 is mounted to the truck frame 14. In this specific example, the controller 104 is mounted inside a cabin of the mixer truck 10, and has a user interface 110 receiving and/or displaying information or alarms in this example. Although the controller 108 is on-truck, and even in-cabin in the illustrated embodiment, it is noted that the controller 108 can be remote from the mixer truck 10 in which case the communication between the controller 108 and the probe 102 can be wireless. In some embodiments, the controller 108 can be omitted.

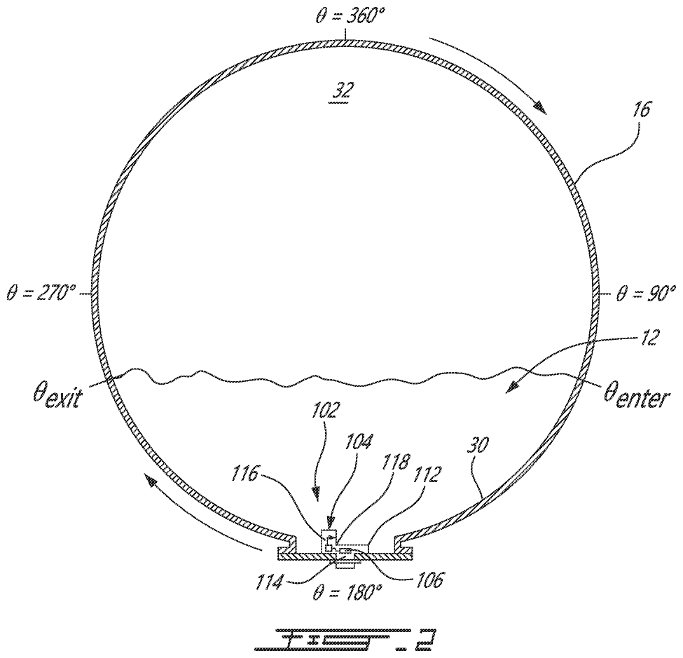

As best seen in FIG. 2, the electromechanical actuator 104 has a probe frame 112 which is fixedly mounted to the drum 16. Accordingly, as the drum 16 is rotated, the electromechanical actuator 104 rotates with it in a circumferential manner across successive circumferential positions. For reference, the probe shown in FIG. 2 is located at an arbitrary circumferential position of 180°, i.e., at the bottom of the drum 16. In this example, the drum 16 may have an opening 114 partially or wholly receiving the probe frame 112. However, the probe frame 112 is itself fixedly mounted to an inner wall 30 of the drum in some other embodiments.

The electromechanical actuator 104 has a moving element 116 which is actuatably mounted to the probe frame 112. Accordingly, upon actuation of the electromechanical actuator 104 with an electrical signal, the electromechanical actuator 104 can convert the electrical energy carried by the electrical signal into mechanical energy through movement of the moving element 116. Examples of such electromechanical actuator 104 can include, but not limited to, a linear movement actuator, a rotational movement motor, a vibratory actuator, a voice coil, a piezoelectric element, a camshaft, a crankshaft and the like.

As shown in this example, the moving element 116 has a fresh concrete interface 118 exposed within the drum 16. It is intended that the fresh concrete interface 118 can be exposed the fresh concrete 12 within the drum 16. Indeed, as the drum 16 rotates over time, the electromechanical actuator 104 can move to some circumferential positions where the fresh concrete interface 118 is immersed in the fresh concrete 12, e.g., when the probe 102 is at the bottom of the drum 16. However, at some other circumferential positions, the fresh concrete interface 118 may be exposed to air, e.g., when the probe 102 is at the top of the drum 16, Accordingly, the fresh concrete interface 118 will always be exposed to a surrounding substance which will at some circumferential positions of the drum 16 be the fresh concrete 12, or air 32 elsewhere. In both cases, upon actuation of the electromechanical actuator 104 with an electrical signal, the fresh concrete interface 118 of the moving element 116 experiences a resistance to movement as it is moved through the surrounding substance within the drum 16.

The measurement unit 106 measures a response of the electromechanical actuator 104 to this resistance (hereinafter “the resistance response”) during the actuation, and generates a response signal based on the measured resistance response. As the resistance response is indicative of the resistance to movement of the fresh concrete interface 118 relative to the surrounding substance, the generated response signal carries information concerning the fresh concrete 12 within the drum 16, if any. Whether the resistance response is greater or weaker upon actuation with a given electrical signal can help monitoring the fresh concrete 12 within the drum 16, as will be described in the following paragraphs.

Example information that can be measured and monitored using the probe 102 are described below:
In some embodiments, the measured resistance response can be used to determine whether the probe 102 is exposed to fresh concrete 12 or to air 32 within the drum 16. For instance, the controller can determine that the probe 102 is exposed to fresh concrete 12 when the measured resistance response is above (or below) a given threshold t1. In contrast, the controller can determine that the probe 102 is exposed to air 32 when the measured resistance response is below (or above) a given threshold t1. In some embodiments, the given threshold t1 can be 0.2 (normalized arbitrary units), in which case it may be determined that the probe 102 is exposed to fresh concrete 12 when the measured resistance response is 0.5 (or 0.1) or to air 32 when the measured resistance response is 0.1 (or 0.5). Depending on the embodiment, alarm(s) may be generated by the controller upon determining that the probe 102 is exposed to air, for instance.
In some embodiments, the measured resistance response can be used to determine whether the probe 102 is exposed to fresh concrete 12, water or air within the drum 16, For instance, the controller can determine that the probe 102 is exposed to fresh concrete 12 when the measured resistance response is above (or below) both first and second thresholds t1 and t2. In contrast, the controller can determine that the probe 102 is exposed to air when the measured resistance response is below (or above) the first and second thresholds t1 and t2. When the measured resistance response is between the first and second thresholds t1 and t2, the controller can determine that the probe 102 is exposed to water, as the density of water is higher than a density of air but lower than a density of fresh concrete. Depending on the embodiment, alarm(s) may be generated by the controller upon determining that the probe 102 is exposed to air or water, for instance.
In some embodiments, the measured resistance response can be monitored as the drum 16 rotates. Therefore, a given number of measured resistance responses can be measured at a corresponding number of timestamps or circumferential positions of the drum 16. The probe 102 may incorporate a probe location sensor such as one or more accelerometers measuring directly or indirectly a circumferential position of the probe 102 at any given time for association to corresponding measured resistance responses. In these embodiments, the circumferential positions at which the probe 102 enters and exits the fresh concrete 12 can be determined by monitoring at which circumferential position the resistance responses, as measured during a single rotation of the drum 16, crosses and crosses back the given threshold t1. However, in some other embodiments, the probe location sensor may be omitted. Regardless of whether a probe location sensor is used, the measured resistance responses can be used to determine at which circumferential positions θₑₙₜₑᵣ and θₑₓᵢₜ of the drum 16 the probe 102 enters and exits the fresh concrete 12. For instance, the controller can determine that, during a given rotation of the drum 16, the measured resistance responses indicate that the probe 102 has remained immersed within the fresh concrete 12 for a given duration Δt. In some embodiments, the duration Δt indicates, for resistance responses measured within a single rotation of the drum 16, a timestamp difference between a timestamp where the measured response crosses the given threshold t1 and another timestamp where the measured responses crosses back the given threshold t1. A curve may be fitted to the measured resistance responses and then solved to obtain its intersection with the given threshold t1 in some other embodiments. The duration Δt may advantageously be normalized based on a rotational speed of the drum 16, if deemed necessary. In some embodiments, such information can then be compared to calibration data to retrieve the circumferential positions θₑₙₜₑᵣ and θₑₓᵢₜ at which the probe 102 enters or exists the fresh concrete 12. Table 1 presented below shows exemplary calibration data for such measurements.

TABLE 1
Exemplary calibration data for determining at which Θₑₙₜₑᵣ
and Θₑₓᵢₜ of the drum 16 the probe 102 enters or exits the fresh concrete 12

| | Circumferential positions |
|---|---|
| Measured duration Δt | Θₑₙₜₑᵣ and Θₑₓᵢₜ |
| [seconds] | [degrees] |
| 0 | none |
| 10 | 90 and 270 |
| 15 | 45 and 315 |

Assuming a rotational speed of the drum of about 3 RPM.

In some embodiments, the circumferential positions θₑₙₜₑᵣ and θₑₓᵢₜ at which the probe 102 enters and exits the fresh concrete 12 can be compared to calibration data to retrieve a volume value indicative of a volume of the fresh concrete 12 within the drum 16. Table 2 presented below shows exemplary calibration data for such measurements.

TABLE 2
Exemplary calibration data for determining the
volume of fresh concrete inside the drum

| Circumferential positions | |
|---|---|
| Θₑₙₜₑᵣ and Θₑₓᵢₜ | Volume value |
| [degrees] | [m³] |
| none | 0 |
| 135 and 225 | 1 |
| 90 and 270 | 3 |

For a drum having a capacity of about 6 m³

In some embodiments, the measured resistance response can be used to determine a property of the fresh concrete 12 to which the fresh concrete interface 118 is exposed. For instance, experiments have confirmed that, assuming that the rotational speed of the drum 16, the amount of concrete above the probe 102, the viscosity, the yield and the temperature of the fresh concrete 12 are constant for the fresh concrete 12 received the drum 16, one can compare the measured resistance response to calibration data in order to determine an air content value indicative of an air content of the fresh concrete 12 within the drum 16. Table 3 presented below shows exemplary calibration data for such measurements.

TABLE 3
Exemplary calibration data for determining the
air content of fresh concrete inside the drum

| Measured resistance | |
|---|---|
| response | Air content value |
| [normalized arbitrary unit] | [%] |
| 0.20 | 0 |
| 0.22 | 2 |
| 0.24 | 4 |

Assuming constant viscosity, yield and temperature

In at least some situations, the fresh concrete 12 can be air-entrained meaning that the fresh concrete 12 contains a significant numbers (e.g., billions) of microscopic air voids per cubic foot. It is known that these air voids can relieve internal pressure inside the fresh concrete 12 by providing tiny chambers within the fresh concrete 12. It was found that these tiny chambers, e.g., their volumes and/or density, may influence the resistance of the fresh concrete 12 to the movement of the fresh concrete interface 118 of the probe 102. It is noted that these tiny chambers can receive water and then expand in freezing temperatures. As a consequence, monitoring air content within a given batch of fresh concrete has been found to be particularly relevant in the context of northern climates where freezing and thawing cycles effects are not insignificant.

In some embodiments, it is predicted that the measured resistance response could also be used to determine other types of property of the fresh concrete 12 to which the fresh concrete interface 118 is exposed. For instance, it is predicted that, assuming that the rotational speed of the drum 16, the amount of concrete above the probe 102, the air content, the yield and the temperature of the fresh concrete 12 are constant for the fresh concrete 12 received the drum 16, one can compare the measured resistance response to calibration data in order to determine a viscosity value indicative of a viscosity of the fresh concrete 12 within the drum 16. Table 4 presented below shows exemplary calibration data for such measurements.

TABLE 4
Exemplary calibration data for determining the
viscosity of fresh concrete inside the drum

| Measured resistance | |
|---|---|
| response | Viscosity value |
| [normalized arbitrary unit] | [arbitrary unit] |
| 0.20 | 1 |
| 0.22 | 2 |
| 0.24 | 3 |

Assuming constant air content, yield and temperature

In some other embodiments, it is predicted that, assuming that the rotational speed of the drum 16, the amount of concrete above, the air content, the viscosity and the temperature of the fresh concrete 12 are constant for the fresh concrete 12 received the drum 16, one can compare the measured resistance response to calibration data in order to determine a yield value indicative of a yield of the fresh concrete 12 within the drum 16. Table 5 presented below shows exemplary calibration data for such measurements.

TABLE 5
Exemplary calibration data for determining
the yield of fresh concrete inside the drum

| Measured response | Yield value |
|---|---|
| [normalized arbitrary unit] | [kPa] |
| 0.20 | 10 |
| 0.22 | 12.5 |
| 0.24 | 15 |

Assuming constant air content viscosity and temperature

Depending on the embodiment of the measurement unit 106, it is noted that the resistance response can be measured as either one or both of a mechanical response and an electrical response.

In the case where the resistance response is mechanical, the measurement unit 106 can have a position sensor measuring a mechanical response of the electromechanical actuator 104 during the actuation. In such a case, the mechanical response typically has an amplitude value indicative of an amplitude of movement of the moving element during the actuation. For a given electrical signal, the amplitude of movement of the moving element 116 may be greater when the surrounding substance is air 32 than when it is fresh concrete 12, as fresh concrete 12 will likely offer more resistance to movement than air 32. Accordingly, the measured response can indicate whether the probe 102 is immersed into the fresh concrete 12 or air 32, for instance.

In the case where the resistance response is electrical, the measurement unit 106 can have an electrical power meter which measures an electrical response of the electromechanical actuator 104 during the actuation. As such, the electrical response can comprise an electrical power value indicative of an electrical power consumed by the electromechanical actuator 104 during the actuation. In at least some circumstances, the electrical power consumed by the electromechanical actuator 104 may be greater when the surrounding substance is fresh concrete than air, as fresh concrete will offer much more resistance to movement than air. It is noted that such proportionality may not be always applicable, as sometimes an oscillatory electrical signal may create a natural resonance response of the fresh concrete interface 118 relative to the surrounding substance, in which case the electromechanical actuator 104 may consume less electrical power than when out-resonance.

It will be appreciated that the given threshold t1 and the calibration data presented above have been presented as examples only. It is clear that depending on whether the measured resistance response is mechanical or electrical, the calibration data can differ. For instance, a measured resistance response being greater than the given threshold t1 can indicate that the probe 102 is exposed to air when the measured resistance response is mechanical, as the fresh concrete interface 118 may move farther away from its rest position for a given electrical signal. However, a measured resistance response being greater than the given threshold t1 can indicate that the probe 102 is exposed to fresh concrete 12 when the measured resistance response is electrical, as moving the fresh concrete interface 118 against the fresh concrete 12 may require more electrical power.

As will be described in the next paragraphs, the probe 102 measures a resistance response that is mechanical. Another probe embodiment measuring an electrical resistance response will be described below with reference to FIG. 9.

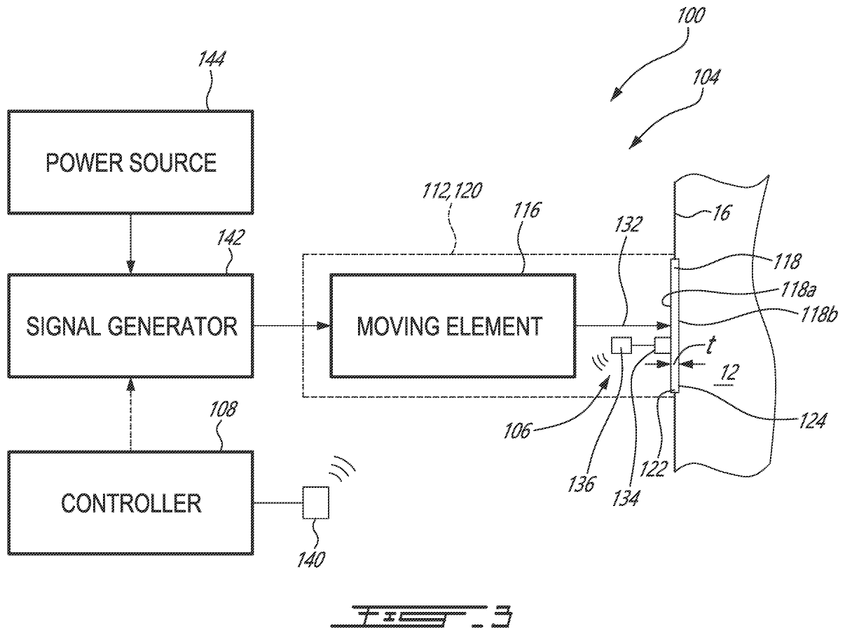

FIG. 3 shows a block diagram of the probe, in accordance with one or more embodiments. As depicted, the moving element 116 of the electromechanical actuator 104 is coupled to the fresh concrete interface 118 via a mechanical coupling 132. An example of mechanical coupling can include, but not limited, to a direct or indirect physical coupling, a spring-loaded coupling, a damped-coupling, and the like.

As shown, the probe frame 112 is provided in the form of a housing 120 inwardly protruding from the inner wall 30 the drum 16. As shown, the housing 130 encloses at least the moving element 116 and the measurement unit 106. In this example, the housing 120 has at least a given wall 122 with an inner side 118a being mechanically coupled to the movement element 116, and an outer side 118b acting as the fresh concrete interface 118. In this way, upon actuation of the electromechanical actuator 104, the moving element 116 moves against the given wall 122 which in turn causes the fresh concrete interface 118 to move against the surrounding substrate inside the drum 16. In such embodiments, the fresh concrete interface 118 is part of the moving element 116 as they are mechanically coupled (e.g., made integral) to one another. In some embodiments, the given wall 122 is provided in the form of a vibratory membrane 124 having a thickness t below a given thickness threshold. For instance, in some embodiments, the vibratory membrane 124 is made of steel and has a thickness t of about 1 mm. In this example, the vibratory membrane 124 is sealingly mounted to the given wall 122 via an urethane seal to allow vibratory movement. In such embodiments, the electromechanical actuator 104 can be analogous to an electroacoustic transducer and the like.

The electrical signal with which the electromechanical actuator 104 is actuated can vary from one embodiment to another. For instance, the electrical signal can have a fixed amplitude, a time-varying amplitude and/or an oscillatory-varying amplitude. When the electrical signal is an oscillatory electrical signal having an amplitude oscillating over time, the resistance response experienced by the fresh concrete interface 118 can oscillate over time correspondingly. The frequency at which the oscillatory-varying amplitude of the electrical signal can vary from an embodiment to another. For instance, the oscillatory electrical signal can have a frequency ranging between about 0 Hz and about 50 kHz, preferably between about 20 Hz and about 20 kHz, and most preferably between about 100 Hz and about 2000 Hz. The frequency can be swept across a given frequency range in some embodiments. In embodiments where the electromechanical actuator 104 is provided in the form of an electroacoustic transducer, the frequency of the electrical signal can vary from 20 Hz to 20 kHz.

In this embodiment, the measurement unit 106 includes one or more mechanical response sensors such as position sensor 134 which is in this case mechanically coupled to the fresh concrete interface 118. Examples of such mechanical response sensors include, but not limited to, magnitude sensor(s), speed sensor(s), accelerometer(s) and the like. These mechanical response sensors can be based on one or more different technologies such as piezoelectric, microelectromechanical systems-(MEMS), optical, capacitive, and inductive, or any combination thereof. The position sensor 134 shown in this example is provided in the form of one or more accelerometers measuring acceleration in one or more orthogonal axes as the fresh concrete interface 118 is being moved against the surrounding substance, and generating a corresponding response signal.

In this specific embodiment, the system 100 has a communication unit 136 receiving the response signal generated by the position sensor 134 and transmitting it towards a communication unit 140 of the controller 108, which is on-truck in this embodiment. Upon receiving the generated response signal, the controller can then send instructions and/or store the generated response signal, for monitoring the fresh concrete 12 right away or later.

As depicted, a signal generator 142 is provided to generate the electrical signal with which the electromechanical actuator 104 is to be actuated. The signal generator 142 is remote from the housing 120 in this embodiment. However, in some other embodiments, the signal generator 142 can be enclosed within the housing 120. The signal generator 142 can be configured to generate electrical signals of different amplitudes, frequencies, durations, and/or of any arbitrary shape. For instance, the electrical signal(s) can have any suitable type of shape including, but not limited to, an impulse shape, a step shape, a harmonic shape and the like. In some embodiments, the controller 108 is communicatively coupled with the signal generator 142 and sends instructions to the signal generator 142 concerning the electrical signal to be generated.

The system 100 can have a power source 144 powering the components. In this example, the power source 144 is remote from the housing 120. However, in some other embodiments, the power source 144 can be enclosed in the housing 120. In such embodiments, the power source 144 can be provided in the form of a battery or battery pack and/or solar panel. It is intended that the power source 144 powers the signal generator 142, the electromechanical actuator 104, the measurement system 106 and/or other components of the system 100. In some other embodiments, the power source 144 is provided in the form of a power supply drawing power from a battery of the mixer truck.

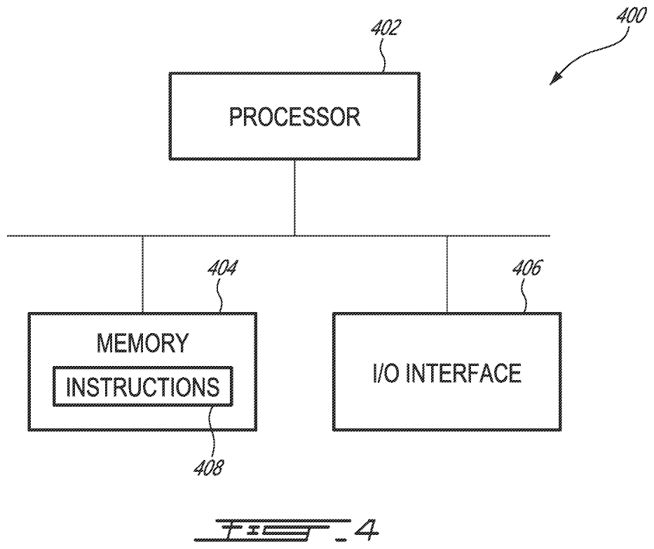

The controller 108 can be provided as a combination of hardware and software components. The hardware components can be implemented in the form of a computing device 400, an example of which is described with reference to FIG. 4.

Referring to FIG. 4, the computing device 400 can have a processor 402, a memory 404, and I/O interface 406. Instructions 408 for monitoring the fresh concrete 12 can be stored on the memory 404 and accessible by the processor 402.

The processor 402 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 404 can include a suitable combination of any type of computer-readable, non-transitory memory that is located either internally or externally such as, for example, random-access memory (RAM), read-only memory (ROM), compact disc read-only memory (CAROM), electro-optical memory, magneto-optical memory, erasable programmable read-only memory (EPROM), and electrically-erasable programmable read-only memory (EEPROM), Ferroelectric RAM (FRAM) or the like.

Each I/O interface 406 enables the computing device 400 to interconnect with one or more input devices, such as mouse(s), keyboard(s), position sensor(s), power meter(s), or with one or more output devices such as a user interface, a non-transitory memory or a remote network. In some embodiments, the user interface is configured to generate alarm(s) based on the generated response signal. It is intended that these alarm(s) may be generated based on a comparison of the generated response signal to reference data, for instance, Such alarm(s) can be visual, auditory, vibratory and the like.

Each I/O interface 406 enables the controller 108 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The computing device 400 described above are meant to be examples only. Other suitable embodiments of the controller 108 can also be provided, as it will be apparent to the skilled reader.

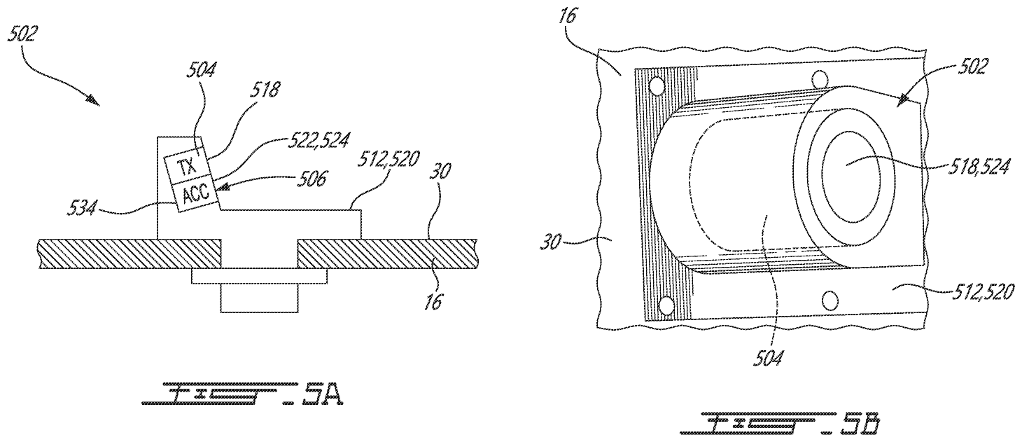

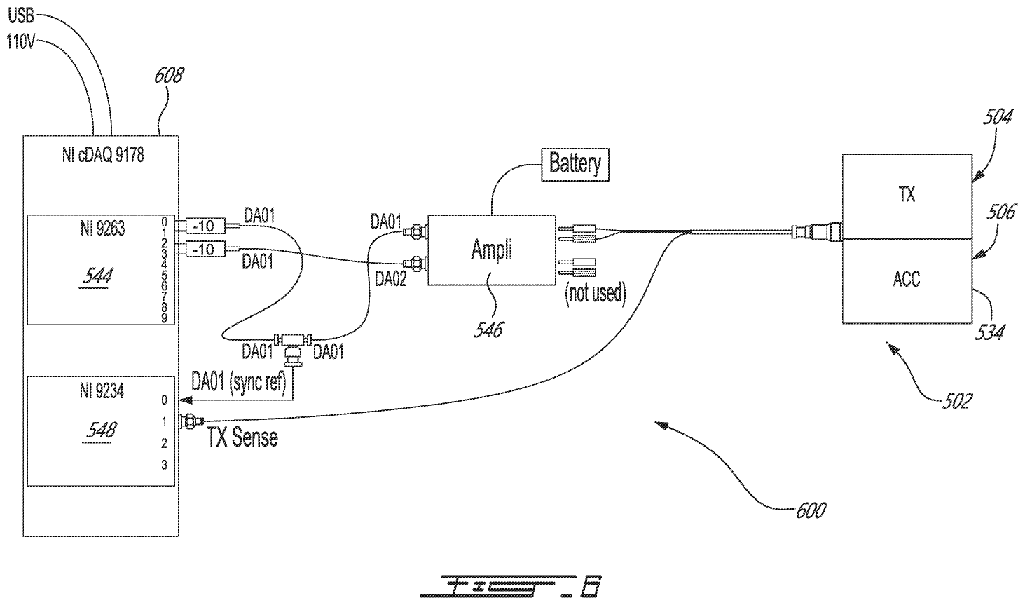

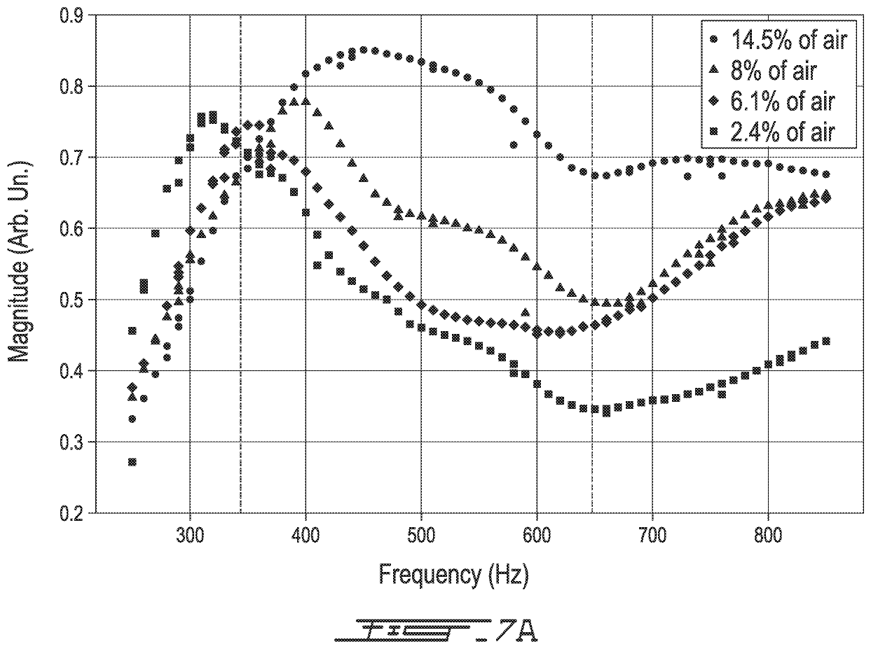

FIGS. 5A and 6B show another example of a probe 502 for monitoring fresh concrete received in a drum 16 of a mixer truck, in accordance with one or more embodiments. As shown, the probe 502 has an electromechanical actuator 504 with a frame 512 mounted to the inner wall 30 within the drum 16 and a moving element actuatably mounted to the frame 512. In this example, the frame 512 is a housing 520 enclosing at least the moving element.

As best shown in FIG. 5A, the housing 520 has a given wall 522 with an inner side mechanically coupled to the moving element and an outer side acting as a fresh concrete interface 118 of the electromechanical actuator 504. The wall is provided in the form of a membrane 524 with a thickness below a given thickness threshold. As such, the fresh concrete interface 118 exposed within the drum will, during use, experience a resistance to movement within the drum 16 upon actuation of the electromechanical actuator 104 with an electrical signal.

As illustrated, the probe 502 has a measurement unit 506 comprising a position sensor 534 measuring a mechanical response of the electromechanical actuator 504. More specifically, in this embodiment, the mechanical response that is measured includes an amplitude value indicative of an amplitude of movement of the moving element during the actuation.

Referring now to FIG. 6, a block diagram of a system 600 incorporating the probe 502 is shown. As depicted in this example, the system 600 includes a controller 608 which is communicatively coupled to the electromechanical actuator 504 and to the measurement unit 506. The controller 608 is provided in the form of a data acquisition system of the type National Instrument cDAQ 9178 in this example. The data acquisition system can be powered using a 110V supply line and has a Universal Serial Bus (USB) port. The data acquisition system in this example has a signal generator 544 of the type National Instrument 9263. An electrical amplifier 546 is used to amplify the electrical signal initially generated by the data acquisition system via electrical cable(s). The electromechanical actuator 504 receives the amplified electrical signal via electrical cable(s). Such components can be integrated on a custom printed circuit board (PCB) that can include any other type of desirable electronic components such as wireless communication units and the like.

As shown, the position sensor 534 generates a response signal which is communicated back to the data acquisition system. More specifically, the data acquisition system has an acousto-vibratory detector 548 of the type National Instrument 9234 which is connected to the position sensor 534 via cable(s).

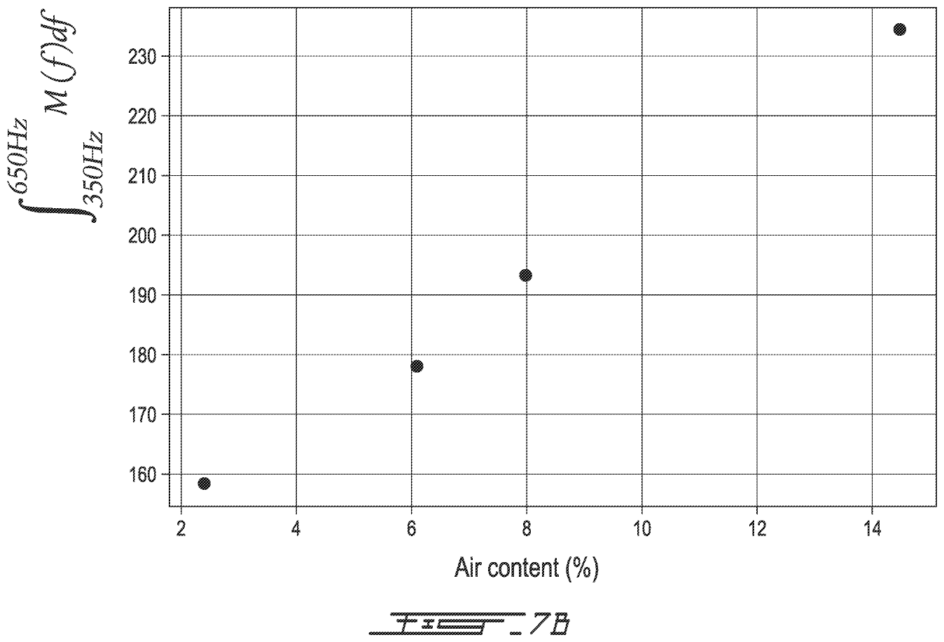

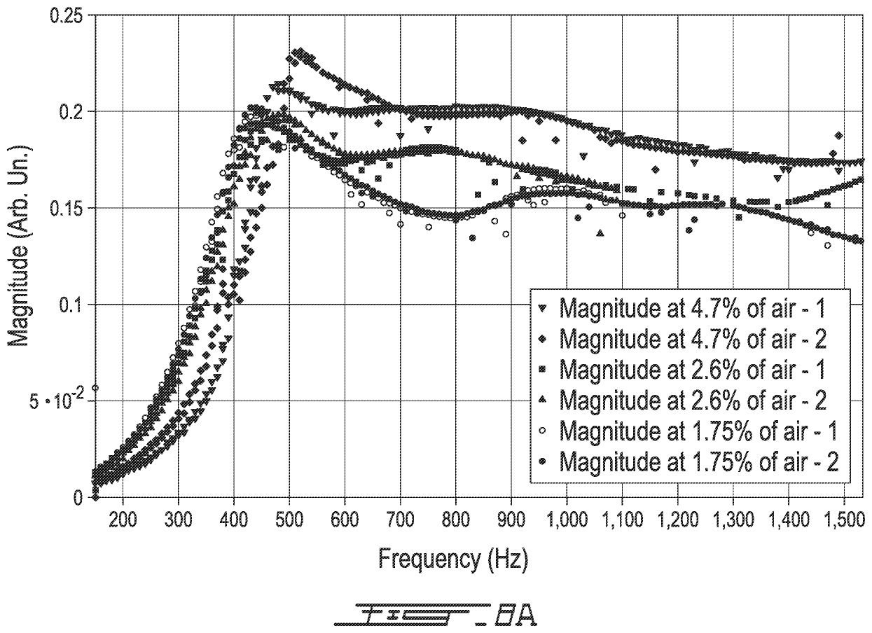

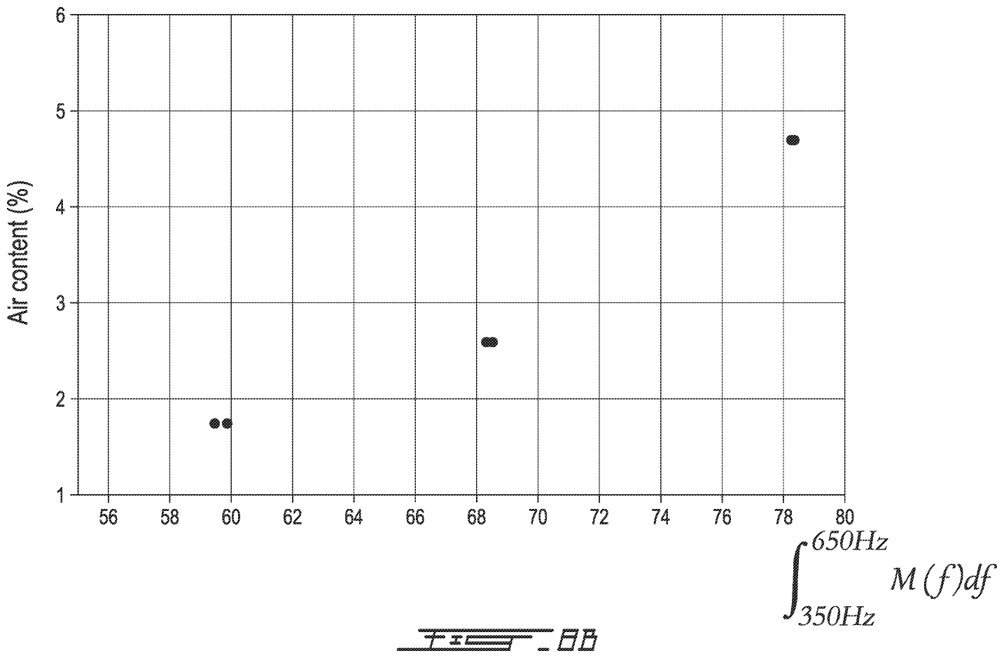

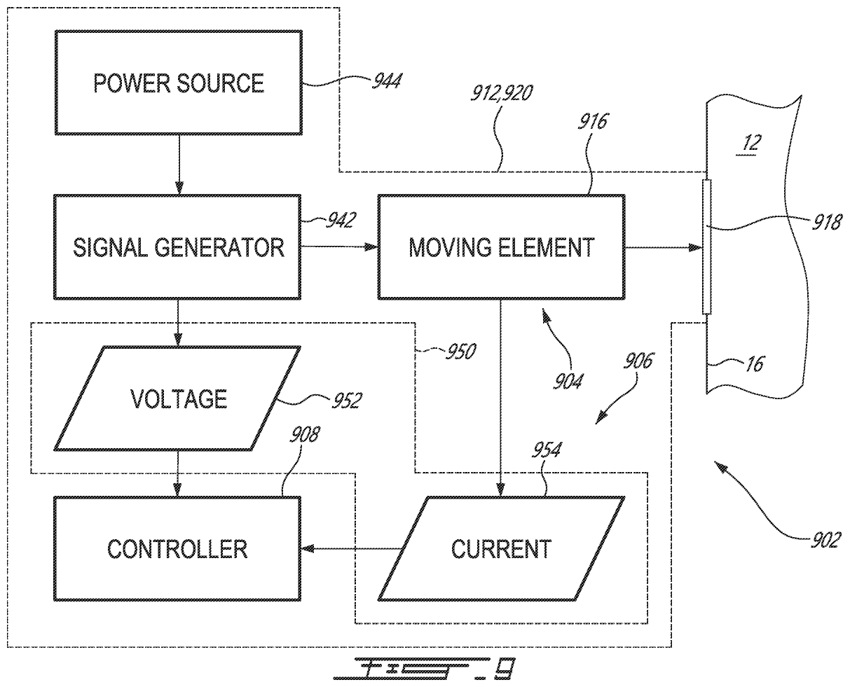

FIGS. 7A through 8B show data measured using the system 600, in one or more experiments.

More specifically, FIG. 7A is a graph showing amplitudes values as measured by the position sensor 534 in the axis of movement of the moving element as function of a frequency of the electrical signal with which the electromechanical actuator 504 is actuated for fresh concrete samples of different air content values. As shown, for each fresh concrete sample, the probe 502 was used to measure a mechanical resistance response of the electromechanical actuator 504 during actuation with an electrical signal having a frequency swept from 250 Hz to 859 Hz. As shown, one can notice that in the frequency band ranging from 350 Hz to 650 Hz, the behaviour of the measured resistance response is relatively proportional to the air content value of the corresponding fresh concrete sample. This relationship is better shown in FIG. 7B which shows integrated values of the curves of FIG. 7A over this frequency band. One can appreciate a relatively linear relationship, which can be used as a basis for calibration data such as those described above.

It is noted that, in this experiment, the fresh concrete samples have properties assumed to be constant except for air content. More specifically, a first fresh concrete sample of given properties (including an air content value of 2.4%) was tested using the probe, then air-entraining adjuvants was added to the first fresh concrete sample to increase the air content to a second air content value of 6.1%, and so forth, for two other iterations. Accordingly, the four fresh concrete samples had similar properties except for their air content. Accordingly, the measured resistance response can be associated to the air content, at least in situations where the other properties of the fresh concrete match with the properties of the fresh concrete used to determine the calibration data.

Although the example above relates to air content, it is predicted that similar conclusions may be reached for other properties such as viscosity, yield and the like.

FIG. 8A shows a graph similar to the one shown in FIG. 7A, but for different fresh concrete samples. Again, a proportional relationship is obtained between the magnitude value and the air content, as emphasized in FIG. 8B.

FIG. 9 shows another example of a probe 902 for monitoring fresh concrete received in a drum 16 of a mixer truck. As shown, the probe 902 has an electromechanical actuator 904 with a frame 912 mounted to the drum 16 and a moving element 916 actuatably mounted to the frame 912. Similarly to the embodiments described above, the moving element 916 has a fresh concrete interface 918 exposed within the drum 16 and experiencing a resistance to movement within the drum 16 upon actuation of the electromechanical actuator 904 with an electrical signal. A measurement unit 906 is provided to measure the resistance response during the actuation, and to generate a corresponding response signal.

In this specific embodiment, instead of measuring a mechanical response, the measurement unit 906 rather measures an electrical response of the actuation. More specifically, the measurement unit 906 has an electrical response sensor, in this case provided in the form of an electrical power meter 950, measuring an electrical response of the electromechanical actuator 904 during the actuation. In this example, the electrical response has an electrical power value indicative of an electrical power consumed by the electromechanical actuator 904 during the actuation. The power meter 950 can be provided in different shape or form. Specifically, in this embodiment, the power meter 950 measures the voltage supplied to the electromechanical actuator using a voltmeter 952 for instance. Moreover, the power meter 950 measures a current that is flowed through the electromechanical actuator 904 using an ammeter 954, for instance. In view of the relation P=VI, wherein P denotes the electrical power value, V denotes the voltage value and I denotes the current value, the controller 908 can monitor the amount of electricity consumed during actuation of the electromechanical actuator 904.

As shown in this embodiment, the frame 912 is provided in the form of a housing 920 enclosing a power source 944, a signal generator 942, the electromechanical actuator 904, the measurement unit 906 and the controller 908.

As can be understood, a given measurement unit may incorporate both the position sensor and the power meter to monitor both the mechanical and the electrical resistance response of the electromechanical actuator. In these embodiments, a property such as air content may be determined using the mechanical resistance response, and proof-reviewed upon determination of the same property but using the electrical resistance response instead, or vice-versa.

As can be understood, the examples described above and illustrated are intended to be exemplary only. For instance, although the system(s) described herein are installed to a mixer truck in this example, the system disclosed herein can be installed on any type of fresh concrete mixers including, but not limited to, stationary mixers, batch mixers, drum type mixers, tilting drum mixers, non-tilting drum mixers, reversing drum mixers, pan type mixers, continuous mixer trucks and the like. The type of measurement unit is not limited to the position sensor and/or to the power meter described above as other types of measurements units can be used as well to monitor a mechanical response and/or an electrical response of the electromechanical actuator in some other embodiments. The scope is indicated by the appended claims.

What is claimed is:

1. A probe for monitoring fresh concrete received in a drum of a fresh concrete mixer, the probe comprising:
an electromechanical actuator having a frame mounted within the drum and a moving element actuatably mounted to the frame, the moving element having a fresh concrete interface exposed within said drum and experiencing a resistance to movement within said drum upon actuation of the electromechanical actuator with an electrical signal; and
a measurement unit measuring a resistance response during said actuation and generating a response signal based on said measured resistance response, the generated response signal comprising monitoring information concerning the fresh concrete within the drum, if any.

2. The probe of claim 1 wherein the frame is a housing enclosing the moving element, the housing having at least a given wall with an inner side mechanically coupled to the moving element and an outer side acting as the fresh concrete interface.

3. The probe of claim 2 wherein the given wall is provided in the form of a membrane having a thickness below a given thickness threshold.

4. The probe of claim 1 wherein the measurement unit has an electrical response sensor measuring an electrical response of said electromechanical actuator during said actuation.

5. The probe of claim 4 wherein the electrical response sensor has an electrical power meter measuring an electrical power value indicative of an electrical power consumed by said electromechanical actuator during said actuation.

6. The probe of claim 1 wherein the measurement unit has a mechanical response sensor measuring a mechanical response of said electromechanical actuator during said actuation.

7. The probe of claim 6 wherein the mechanical response sensor has a position sensor measuring an amplitude value indicative of an amplitude of movement of said moving element during said actuation.

8. The probe of claim 1 further comprising a controller communicatively coupled to the measurement unit, the controller having a processor and a non-transitory memory having stored thereon instructions that when executed by the processor performs the step of monitoring the fresh concrete received in the drum based on said generated response signal.

9. The probe of claim 8 wherein said actuation and measurement are performed a plurality of times during at least a rotation of the drum, said monitoring including determining a volume of the fresh concrete inside the drum based on said resistance responses experienced during the at least the rotation of the drum.

10. The probe of claim 8 wherein said monitoring includes determining a rheological property of said fresh concrete, said rheological property being selected in a group of rheological properties including viscosity, yield and slump.

11. The probe of claim 8 wherein said monitoring includes determining a physical property of said fresh concrete, said physical property being selected in a group of physical properties including air content and density.

12. The probe of claim 8 wherein said monitoring is based on calibration data pertaining to different resistance responses as function of different properties of the fresh concrete.

13. The probe of claim 1 wherein said electrical signal is an oscillatory electrical signal having an amplitude oscillating over time, the resistance response experienced by the fresh concrete interface oscillating over time during said actuation with said oscillatory electrical signal.

14. The probe of claim 13 wherein said oscillatory electrical signal has a frequency ranging between about 20 Hz and about 20 kHz.

15. The probe of claim 1 wherein the fresh concrete mixer is a mixer truck.

16. A method of monitoring fresh concrete received in a drum of a fresh concrete mixer, the method comprising:
exposing a fresh concrete interface within said drum;
mechanically coupling a moving element of an electromechanical actuator to said fresh concrete interface;
actuating the electromechanical actuator with an electrical signal, said actuating including moving said moving element relative to the fresh concrete interface, said moving element thereby experiencing a resistance to movement via said fresh concrete interface;
measuring a resistance response during said actuating and generating a response signal based on said measured resistance response, the generated response signal comprising monitoring information concerning the fresh concrete within the drum, if any.

17. The method of claim 16 wherein said measuring the resistance response includes measuring an electrical response of said electromechanical actuator during said actuation.

18. The method of claim 17 wherein said measuring the electrical response includes measuring an electrical power value indicative of an electrical power consumed by said electromechanical actuator during said actuation.

19. The method of claim 16 wherein said measuring the resistance response includes measuring a mechanical response of said electromechanical actuator during said actuation.

20. The method of claim 19 wherein said measuring the mechanical response includes measuring an amplitude value indicative of an amplitude of movement of said moving element during said actuation.

21. The method of claim 16 further comprising monitoring said fresh concrete based on the generated response signal.

22. The method of claim 21 wherein said actuating and said measuring are performed a plurality of times during at least a rotation of the drum, said monitoring including determining a volume of the fresh concrete inside the drum based on said resistance responses experienced during the at least the rotation of the drum.

23. The method of claim 21 wherein said monitoring includes determining a rheological property of said fresh concrete, said rheological property being selected in a group of rheological properties including viscosity, yield and skimp.

24. The method of claim 21 wherein said monitoring includes determining a physical property of said fresh concrete, said physical property being selected in a group of physical properties including air content and density.

25. The method of claim 16 wherein said electrical signal is an oscillatory electrical signal having an amplitude oscillating over time, said actuating including moving said moving element against the fresh concrete interface in at least a back and forth sequence.

26. The method of claim 16 wherein the fresh concrete mixer is a mixer truck.
